(19) United States
(12) Patent Application Publication
(10) Pub. No.: US 2018/0125549 A1
(43) Pub. Date: May 10, 2018

(54) METHOD OF TREATING A DISTAL RADIUS FRACTURE

(71) Applicant: Universitaet Rostock, Rostock (DE)

(72) Inventors: Georg Gradl, Boergerende (DE); Hans-Werner Stedtfeld, Rostock (DE)

(21) Appl. No.: 15/862,090

(22) Filed: Jan. 4, 2018

Related U.S. Application Data
(63) Continuation of application No. 13/380,133, filed on Dec. 22, 2011.

(30) Foreign Application Priority Data
Jun. 25, 2009 (DE) 10 2009 030 622.6

(51) Int. Cl. A61B 17/80

(52) U.S. Cl. CPC A61B 17/8061; A61B 17/8095; A61B 17/8028

(57) ABSTRACT

A method of treating a distal radius fracture that produces a bone fragment at a radius shaft, including the steps of: obtaining: a) an osteosynthesis plate with a long, proximal leg and a short, distal leg; and b) a distancing element; fastening the short, distal leg to the bone fragment with the distancing element acting between the long, proximal leg and the radius shaft so that the distancing element maintains the long, proximal leg at a specific angle with respect to the radius shaft; removing the distancing element from between the long, proximal leg and the radius shaft; pressing the long, proximal leg towards the radius shaft to thereby reposition the bone fragment relative to the radius shaft; and fastening the long, proximal leg to the radius shaft.

### CROSS-REFERENCE TO RELATED APPLICATIONS

This application is a continuation of U.S. application Ser. No. 13/380,133, filed Dec. 22, 2011, which is a national stage of PCT/EP2010/058910, filed Jun. 23, 2010.

### BACKGROUND OF THE INVENTION

### Field of the Invention

The invention relates to a repositioning device and a method for the treatment of distal radius fractures by means of an osteosynthesis plate.

### Background Art

The use of angle-stable plates is known for the treatment of fractures of the forearm distant from the body (spoke bone, radius). A problem here is to obtain an adequate repositioning of the fracture fragments. The outcome should be to restore the human anatomy before the fall.

The anatomical position in the frontal plane with an angle of 25° steepness and approx. 1-5 mm additional length with respect to the ulna at the level of the distal radioulnar joint is decisive for the distal spoke bone (radius) distant from the body. In the sagittal plane, the articular surface of the radius is tilted by approx. 10° in the palmar direction (palm of the hand side). In a typical fracture of the radius, there is a dislocation of the distal fracture fragment out of this anatomical position of around 20-50° in the direction of the back of the hand (dorsal). This dislocation has to be corrected by the treating surgeon. However, this is usually not completely successful. An angle of 0° to 5°, in many cases only a repositioning angle of 0°, is achieved with a conventional operation. In this case, the distal fracture fragment in the sagittal plane is fixed only vertical to the radius shaft.

A T-shaped osteosynthesis plate is previously known from US 2005/0065522 A1. When it is used for an osteotomy, the short leg is screwed by means of bone screws to the distal end of the bone, the subsequent fracture segment, the long leg standing away at an angle from the proximal bone near the body. The bone proximal to the distal end is then sawn off and the plate is swiveled through the angle by means of its long leg to the bone near the body and screwed to the proximal bone near the body by means of bone screws introduced through the long leg into the proximal bone near the body.

The known T-shaped osteosynthesis plate can be used not only in connection with an osteotomy, but also in connection with bone fractures, as has been described at the outset. In the case of a typical fracture of the distal radius distant from the body, the dislocation of the distal fracture fragment has to be corrected by the treating surgeon. However, this is not usually completely successful with the known T-shaped osteosynthesis plate.

The problem underlying the invention, therefore, is to improve the repositioning device and the method for the treatment of distal radius fractures by means of a T-shaped osteosynthesis plate, in such a way that the required tilting of the articular surface in the palmar direction of the employed osteosynthesis plate can be carried out with angular precision.

### SUMMARY OF THE INVENTION

In order to solve this problem, the invention provides a repositioning device, wherein the lower surface of the long, proximal leg of the T-shaped plate facing the proximal bone or radius shaft is provided with a spacer in order to keep the long leg at a specific angle with respect to the proximal bone near the body.

For this purpose, the spacer beneath the long leg of the T-shaped plate is adjusted to achieve the required angle. When the long leg is then placed with its lower surface, with the interposition of the spacer, on the proximal bone near the body, the short leg of the T-shaped plate already lying with its underside on the dislocated fracture fragment, the T-shaped plate is first fixed distally with the bone screws. The long leg of the T-shaped plate does not therefore lie directly on the proximal bone, but rather stands several millimetres away. An angle between 5° and 20° thus arises between the distal fracture fragment fixed in a neutral position (neutral with respect to the shaft axis of the bone) and the T-shaped plate. If the spacer is now removed by the surgeon and the T-shaped plate is pressed with the long leg onto the proximal bone, the distal fracture fragment fastened to the T-shaped plate is tilted in the palmar direction by the angle of the previously introduced spacer. The anatomy of the distal radius is thus restored in a straightforward manner with a 10° palmar tilt in the sagittal plane, i.e. the remaining 10° tilt of the articular surface in the palmar direction is achieved again together with the T-shaped plate.

In a further embodiment of the invention, the spacer is a distancing element in the form of a wedge, cylinder or parallelepiped that can be disposed on the lower surface of the long leg facing the proximal bone near the body. These distancing elements have to be adapted in their size to the desired angle before insertion in order to achieve a specific, desired angle between the long leg and the proximal bone.

According to the invention, the wedge has an angle of 5° to 20° between its wedge surfaces resting on the proximal bone near the body and on the lower surface of the long leg facing said proximal bone. Furthermore, the wedge can be fastened detachably on the contact side of the long leg facing the proximal bone near the body. With the spacer according to the invention, in this case a wedge, an anatomical repositioning of at least approx. 15° is achieved.

In a further embodiment of the invention, the spacer or the distancing element is a distancing screw that can be screwed in the long leg, said distancing screw comprising a contact face for the proximal bone near the body. Furthermore, the distancing screw can comprise a head thread and a smooth shank. The distancing screw that can be screwed in the long leg enables easy adaptation of the angle between the long leg and the proximal bone to the requirements of the individual application.

The invention also relates to a method for the treatment of distal radius fractures by means of the T-shaped osteosynthesis plate of the repositioning device. The short, distal leg of the T-shaped plate is first screwed to the distal fracture fragment, the long, proximal leg of the T-shaped plate being held spaced apart by means of the spacer at a specific angle with respect to the proximal bone near the body, and finally after removal of the spacer the long leg of the T-shaped plate is pressed onto the proximal bone near the body and is fastened there, as a result of which the distal fracture fragment fastened to the T-shaped plate is tilted by the adjusted angle in the palmar direction.

In one form, the invention is directed to a method of treating a distal radius fracture that produces a bone fragment at a radius shaft. The method includes the steps of: obtaining: a) an osteosynthesis plate with a long, proximal leg and a short, distal leg; and b) a distancing element; fastening the short, distal leg to the bone fragment with the distancing element acting between the long, proximal leg and the radius shaft so that the distancing element maintains the long, proximal leg at a specific angle with respect to the radius shaft; removing the distancing element from between the long, proximal leg and the radius shaft; pressing the long, proximal leg towards the radius shaft to thereby reposition the bone fragment relative to the radius shaft; and fastening the long, proximal leg to the radius shaft,

In one form, the short, distal leg has an opening therein. The step of fastening the short, distal leg to the bone fragment involves directing a fastener through the opening in the short, distal leg and into the bone fragment.

In one form, the long, proximal leg has an opening therein. The step of fastening the long, proximal leg to the radius shaft involves directing a fastener through the opening in the long, proximal leg and into the radius shaft.

In one form, the long, proximal leg has a surface that engages the radius shaft with the long, proximal leg fastened to the radius shaft. The step of obtaining a distancing element involves obtaining a distancing element that is configured to maintain the surface of the long, proximal leg at an angle of 5-20° with respect to the radius shaft with the distancing element acting between the long, proximal leg and the radius shaft.

In one form, the short, distal leg has a surface that engages the bone fragment with the short, distal leg fastened to the bone fragment. The step of obtaining an osteosynthesis plate involves obtaining an osteosynthesis plate wherein the surfaces on the long, proximal leg and short, distal leg together make an obtuse angle.

In one form, the step of removing the distancing element from between the long, proximal leg and the radius shaft involves fully separating the distancing element from the osteosynthesis plate.

In one form, the distancing element and osteosynthesis plate are fully separable from each other. The method of treating a distal radius fracture further includes the step of directing a fastener through the long, proximal leg and distancing element and into the radius shaft to maintain the distancing element between the long, proximal leg and the radius shaft.

In one form, the method further includes the step of removing the fastener to allow the distancing element to be separated from the osteosynthesis plate before the long, proximal leg is fastened to the radius shaft.

In one form, the distancing element has oppositely facing surfaces that are angled with respect to each other. One of the oppositely facing surfaces engages the long, proximal leg and the other of the oppositely facing surfaces engages the radius shaft with the distancing element maintaining the long, proximal leg at the specific angle.

In one form, the step of obtaining an osteosynthesis plate involves obtaining a T-shaped osteosynthesis plate.

In one form, with the long, proximal leg fixed to the radius shaft the surface of the short, distal leg makes an angle of at least 15° with respect to the surface of the long, proximal leg.

In one form, the distancing element has oppositely facing surfaces that are substantially parallel with respect to each other. One of the oppositely facing surfaces engages the long, proximal leg and the other of the oppositely facing surfaces engages the radius shaft with the distancing element maintaining the long, proximal leg at the specific angle.

In one form, the step of obtaining a distancing element involves obtaining a distancing element that is configured to be advanced relative to the long, proximal leg and against the radius shaft to thereby selectively change the specific angle.

In one form, the step of obtaining a distancing element involves obtaining a distancing element that is threadably engaged with the long, proximal leg. The method further includes the step of turning the distancing element relative to the long, proximal leg to thereby change the specific angle.

In one form, the distancing element has a shank that is unthreaded.

In one form, the distancing element has a threaded shank.

In one form, the distancing element has a shank with a blunt free end that abuts to the radius shaft with the distancing element maintaining the long, proximal leg at the specific angle.

In one form, the method further includes the step of separating the distancing element from the long, proximal leg before fastening the long, proximal leg to the radius shaft.

In one form, the long, proximal leg has a distal end adjacent the short, distal leg and a free, proximal end. The distancing element is closer to the free proximal end than the distal end with the distancing element maintaining the long, proximal leg at the specific angle.

In one form, the step of fastening the short, distal leg involves directing a plurality of fasteners through the short, distal leg and into the bone fragment. The step of fastening the long, proximal leg involves directing a plurality of fasteners through the long, proximal leg and into the radius shaft.

### BRIEF DESCRIPTION OF THE DRAWINGS

The repositioning device according to the invention for the treatment of distal radius fractures by means of a T-shaped osteosynthesis plate is explained in greater detail below with the aid of several embodiments. In the figures:

FIG. 1A shows the distal radius of a distal radioulnar joint in the frontal plane,

FIG. 1B shows the distal radius according to FIG. 1A in the sagittal plane,

FIG. 2A shows a distal radius with dislocation of the distal fracture fragment in the dorsal direction,

FIG. 2B shows the distal radius fracture according to FIG. 2A post-operatively with a T-shaped osteosynthesis plate according to the prior art,

FIG. 3A shows the distal radius fracture according to FIG. 2A with a T-shaped osteosynthesis plate and with a spacer in the form of a wedge under the long leg of the T-shaped osteosynthesis plate according to the first embodiment of the invention,

FIG. 3B shows the distal radius fracture according to FIG. 3A post-operatively after the removal of the wedge under the long leg of the T-shaped osteosynthesis plate in the first embodiment of the invention,

FIG. 4A shows the second embodiment of the invention, modified from the first embodiment according to FIG. 3A, with a screwed wedge under the long leg of the T-shaped plate,

FIG. 4B shows the fourth embodiment of the invention, modified from the third embodiment according to FIG. 4A, with a distancing screw as a spacer, and

FIGS. 5A to 5E show further embodiments of spacers in the form of a wedge, a cylinder, a parallelepiped, a distancing screw and a screw pin.

### DETAILED DESCRIPTION OF THE PREFERRED EMBODIMENT

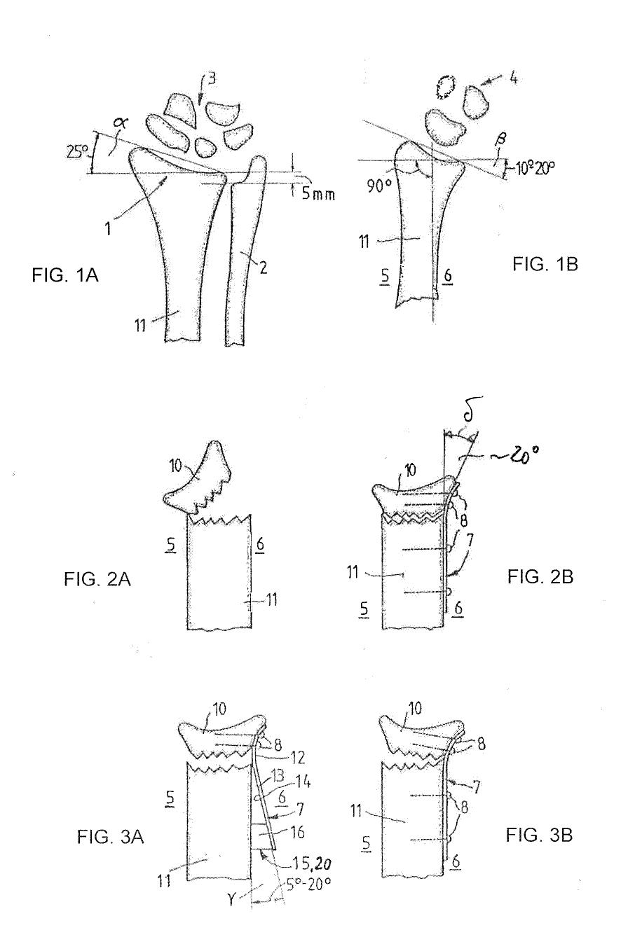

FIG. 1A and 1B show a patient's forearm facing outwards from the body (spoke bone, radius) with radius 1, ulna 2, metacarpus 3 and metacarpal bone 4. The problem in the treatment of a fracture of the forearm distant from the body (FIG. 2A) is to achieve adequate repositioning of fracture fragments 10. The aim is to restore the human anatomy before a fall (FIG. 1A, 1B) after a fracture (FIG. 2A). The anatomical position in the frontal plane (FIG. 1A) with an angle α of 25° steepness and approx. 1-5 mm additional length (FIG. 1A) with respect to ulna 2 at the level of the distal radioulnar joint is decisive for distal spoke bone or radius 1 distant from the body. In the sagittal plane according to FIG. 1B, the articular surface of radius 1 is tilted by an angle β of approx. 10° in the palmar direction (palm of the hand side) according to reference number 6 in FIG. 1B and 2A. In a typical fracture of radius 1, there is a dislocation of distal fracture fragments 10 out of this anatomical position by 20 to 50° in the direction of the back of the hand (dorsal) according to reference number 5 in FIG. 2A. This dislocation has to be corrected by the treating surgeon. However, this is usually not completely successful. A repositioning of 0° is all that is achieved in many cases. In this case, distal fracture fragment 10 in the sagittal plane according to FIG. 2B can be fixed only normal to the radius shaft 11.

This previously known repositioning takes place by means of a T-shaped osteosynthesis plate 7, in short a T-shaped plate 7, the short and long legs 12, 13 whereof are screwed according to FIG. 2B to fracture fragments 10 and radius shaft 11 by means of bone screws 8 engaging through openings 9 in T-shaped plate 7 in the palmar direction according to reference number 6, radius 1 occupying the position shown in FIG. 2B with respect to radius shaft 11. Bending ankle δ between short and long leg 12 and 13 respectively amounts to approx. 20° (FIG. 2B).

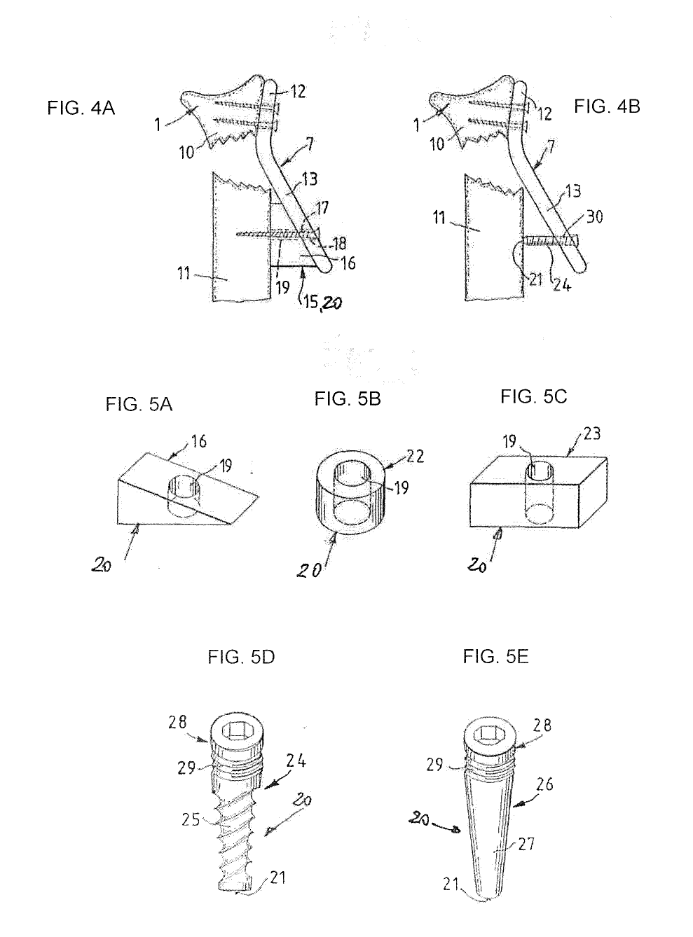

In order to make it easier for the surgeon to achieve the remaining 10° tilt of the articular surface in the palmar direction according to reference number 6 together with employed T-shaped plate 7, lower face 14 of long, proximal leg 13 facing the proximal bone or radius shaft 11 is provided according to the present invention with a spacer 15 in the form of a wedge 16 in order to keep long leg 13 at a specific angle γ of between 5° and 20° by means of its wedge faces. Wedge 16 can be held in particular by means of a bone screw 17, which passes through holes 18 and 19 in long leg 13 and in wedge 16 (FIG. 4A). When pre-formed T-plate 7 is placed on bone or radius shaft 11, after short leg 12 of plate 7 has already been fastened distally on dislocated fracture fragments 10 by means of bone screws 8, long leg 13 of T-plate 7 does not therefore lie directly on proximal bone or radius shaft 11, but rather stands away by several millimetres, depending on the size of angle γ. An angle γ between 5° and 20° thus results between distal fracture fragment 10 fixed in a neutral position (neutral with respect to the shaft axis of proximal bone or radius shaft 11) and T-plate 7. If wedge 16 is now removed by the surgeon and T-plate 7 is pressed with its long leg 13 on proximal bone or radius shaft 11, distal fracture fragment 10 fastened to short leg 12 of T-shaped plate 7 is tilted through angle γ of previously introduced and now removed wedge 16 in the palmar direction (reference number 6) according to FIG. 3B. The anatomy of distal radius 1 with a 10° palmar tilt in the sagittal plane (FIG. 3B) is thus reproduced in a straightforward manner. Spacer 15, in this case wedge 16, leads to an anatomical repositioning of at least approx. 15°.

Spacer 15 forms a distancing element 20 disposed on lower surface 14 of long leg 13 facing proximal bone or radius shaft 11 near the body. According to FIG. 3A and 4A, this can be a wedge 16, which is fastened according to FIG. 4A by means of a bone screw 17, which passes through holes 18 and 19 in long leg 13 of T-shaped plate 7 and wedge 16 (FIG. 5A) and which is screwed into proximal bone or radius shaft 11. According to FIG. 5B, however, distancing element 20 can also be a cylinder 22 or according to FIG. 5C a parallelepiped 23, which in each case are provided like wedge 16 with holes 19 for the fixing.

The long leg 13 having lengthwise proximal and distal ends. The spacer 15 and plate 7 are configured so that with the short leg 12 operatively fastened, the spacer 15 produces a wedging force in the plate 7 at a location closer to the proximal end than the distal end.

According to FIG. 4B in combination with FIG. 5D or 5E, distancing element 20 can also be a distancing screw 24 with a shank thread 25 or a screw pin 26 with a smooth shank 27, which in each case are provided with blunt contact faces 21 for proximal bone or radius shaft 11 near the body. Distancing screw 24 and screw pin 26 both have a thread 29 on the head 28, with which they can be screwed into a threaded hole 30 in long leg 13 of T-shaped plate 7 and connected thereto.

By means of distancing screw 24 or screw pin 26 of differing length, arbitrary distances between long leg 13 of T-shaped plate 7 and proximal bone or radius shaft 11 and therefore arbitrary angles γ can be adjusted. It can thus be seen that a spacer 16 is provided as a distancing element 20, which is not necessarily a solid wedge 16, but rather other geometrical figures can also be used.

The method for the treatment of distal radius fractures by means of T-shaped osteosynthesis plate 7 of the repositioning device described above is characterised in that short, distal leg 13 of T-shaped plate 7 is rigidly screwed to distal fracture fragment 10, that long, proximal leg 13 of T-shaped plate 7 is held spaced apart by means of spacer 15 or distancing element 20 at a specific angle γ between 5° and 20° with respect to proximal bone or radius shaft 11 facing inwards towards the body and that, after removal of spacer 15 or distancing element 20, long leg 13 of T-shaped plate 7 is pressed onto proximal bone or radius shaft 11 and is fastened there by means of bone screws 8, as a result of which distal fracture fragment 10 fastened to T-shaped plate 7 is tilted by adjusted angle γ in the palmar direction according to reference number 6 (FIG. 3A, 3B).

The anatomical adaptation of T-shaped plate 7, which has an angulation or an angle δ of approx. 20° of short leg 12 of T-shaped plate 7 with respect to long leg 13, rules out the possibility of this pre-bending alone also leading automatically to a corresponding fracture repositioning. The 0° position of the articular surface is on the contrary reached with the angular T-shaped osteosynthesis plates previously known in the prior art, as has already been mentioned in the introduction. As a result of the inventive embodiment of angled T-shaped plate 7, said angulation automatically resulting from the plate pre-bending through angle δ and the use of a spacer 15 or distancing element 20, e.g. in the form of wedge 16, the fracture is “forced” into the required tilt. With the angulation of T-shaped plate 7 alone, this is only possible if T-shaped plate 7 somehow “is held in air” during the operation. This is accompanied by the risk of T-shaped plate 7 then not lying orthograde on bone shaft or radius shaft 11, but being displaced laterally. The surgeon cannot possibly bring a T-shaped plate 7 held by hand in the air subsequently up to bone or radius shaft 11 with the required precision. Moreover, a defined spacing is thus also not complied with that subsequently permits a predictable tilt of fracture fragment 10. This purely “manual” precision is not sufficient and is fundamentally changed by the repositioning device according to the invention and the method performed with said repositioning device.

T-shaped osteosynthesis plate 7 alone is not a repositioning device for the treatment of distal radius fractures, but rather a pure support plate, which is adapted by its angulation to the anatomy and is placed on a bone preset by other fixing elements such as Kirschner wires and already prefixed anatomically and, due to its angulation, does not then displace the latter again during the fixing.

There follows a comparison of the chronological sequence in the case of

A: a normal operation with a T-shaped osteosynthesis plate according to the prior art:
1. fracture presentation,
2. setting—i.e. resetting,
3. temporarily holding the repositioning with wires,
4. obtaining a desired fracture position,
5. when the desired fracture position has been reached, fitting of the T-shaped osteosynthesis plate,
6. fixing of the T-shaped plate.

with

B: the chronological sequence with an osteosynthesis plate in the first embodiment of the invention with a wedge:
1. fracture presentation,
2. setting—i.e. resetting,
3. holding the repositioning with wires,
4. then—irrespective of the obtained repositioning—fitting of the T-shaped plate with the wedge,
5. fixing of the plate with the repositioning wedge on the bone or radius shaft,
6. fixing of the fracture,
7. removal of the wedge and now—by bringing the plate up to the bone—obtaining the desired repositioning.

This comparison of the repositioning procedures according to the prior art and according to the invention shows that the chronological sequence in a normal operation agrees with the chronological sequence with the repositioning device according to the invention only in steps 1 to 3. The sequence with the use of the repositioning device according to the invention then takes, with steps 4 to 7, a different procedural route from the previously known method which then comprises steps 4 to 6.

### LIST OF REFERENCE NUMBERS

1 radius
2 ulna
3 metacarpus
4 metacarpal bone
5 dorsal
6 palmar
7 plate
8 bone screw
9 opening
10 fracture fragment
11 radius shaft
12 short leg
13 long leg
14 lower surface
15 spacer
16 wedge
17 bone screw
18 hole
19 hole
20 distancing element
21 contact face
22 cylinder
23 parallelepiped
24 distancing screw
25 shank thread
26 screw pin
27 shank
28 head
29 thread
30 threaded hole

The foregoing disclosure of specific embodiments is intended to be illustrative of the broad concepts comprehended by the invention.

1. A method of treating a distal radius fracture that produces a bone fragment at a radius shaft, the method comprising the steps of:
obtaining: a) an osteosynthesis plate with a long, proximal leg and a short, distal leg; and b) a distancing element;
fastening the short, distal leg to the bone fragment with the distancing element acting between the long, proximal leg and the radius shaft so that the distancing element maintains the long, proximal leg at a specific angle with respect to the radius shaft;
removing the distancing element from between the long, proximal leg and the radius shaft;
pressing the long, proximal leg towards the radius shaft to thereby reposition the bone fragment relative to the radius shaft; and
fastening the long, proximal leg to the radius shaft.

2. The method of treating a distal radius fracture according to claim 1 wherein the short, distal leg has an opening therein and the step of fastening the short, distal leg to the bone fragment comprises directing a fastener through the opening in the short, distal leg and into the bone fragment.

3. The method of treating a distal radius fracture according to claim 1 wherein the long, proximal leg has an opening therein and the step of fastening the long, proximal leg to the radius shaft comprises directing a fastener through the opening in the long, proximal leg and into the radius shaft.

4. The method of treating a distal radius fracture according to claim 1 wherein the long, proximal leg has a surface that engages the radius shaft with the long, proximal leg fastened to the radius shaft and the step of obtaining a distancing element comprises obtaining a distancing element that is configured to maintain the surface of the long, proximal leg at an angle of 5-20° with respect to the radius shaft with the distancing element acting between the long, proximal leg and the radius shaft.

5. The method of treating a distal radius fracture according to claim 4 wherein the short, distal leg has a surface that engages the bone fragment with the short, distal leg fastened to the bone fragment and the step of obtaining an osteosynthesis plate comprises obtaining an osteosynthesis plate wherein the surfaces on the long, proximal leg and short, distal leg together make an obtuse angle.

6. The method of treating a distal radius fracture according to claim 1 wherein the step of removing the distancing element from between the long, proximal leg and the radius shaft comprises fully separating the distancing element from the osteosynthesis plate.

7. The method of treating a distal radius fracture according to claim 1 wherein the distancing element and osteosynthesis plate are fully separable from each other and further comprising the step of directing a fastener through the long, proximal leg and distancing element and into the radius shaft to maintain the distancing element between the long, proximal leg and the radius shaft.

8. The method of treating a distal radius fracture according to claim 7 further comprising the step of removing the fastener to allow the distancing element to be separated from the osteosynthesis plate before the long, proximal leg is fastened to the radius shaft.

9. The method of treating a distal radius fracture according to claim 8 wherein the distancing element has oppositely facing surfaces that are angled with respect to each other, one of the oppositely facing surfaces engaging the long, proximal leg and the other of the oppositely facing surfaces engaging the radius shaft with the distancing element maintaining the long, proximal leg at the specific angle.

10. The method of treating a distal radius fracture according to claim 1 wherein the step of obtaining an osteosynthesis plate comprises obtaining a T-shaped osteosynthesis plate.

11. The method of treating a distal radius fracture according to claim 5 wherein with the long, proximal leg fixed to the radius shaft the surface of the short, distal leg makes an angle of at least 15° with respect to the surface of the long, proximal leg.

12. The method of treating a distal radius fracture according to claim 8 wherein the distancing element has oppositely facing surfaces that are substantially parallel with respect to each other, one of the oppositely facing surfaces engaging the long, proximal leg and the other of the oppositely facing surfaces engaging the radius shaft with the distancing element maintaining the long, proximal leg at the specific angle.

13. The method of treating a distal radius fracture according to claim 1 wherein the step of obtaining a distancing element comprises obtaining a distancing element that is configured to be advanced relative to the long, proximal leg and against the radius shaft to thereby selectively change the specific angle.

14. The method of treating a distal radius fracture according to claim 13 wherein the step of obtaining a distancing element comprises obtaining a distancing element that is threadably engaged with the long, proximal leg and further comprising the step of turning the distancing element relative to the long, proximal leg to thereby change the specific angle.

15. The method of treating a distal radius fracture according to claim 14 wherein the distancing element has a shank that is unthreaded. 16, The method of treating a distal radius fracture according to claim 14 wherein the distancing element has a threaded shank.

17. The method of treating a distal radius fracture according to claim 14 wherein the distancing element has a shank with a blunt free end that abuts to the radius shaft with the distancing element maintaining the long, proximal leg at the specific angle.

18. The method of treating a distal radius fracture according to claim 14 further comprising the step of separating the distancing element from the long, proximal leg before fastening the long, proximal leg to the radius shaft.

19. The method of treating a distal radius fracture according to claim 1 wherein the long, proximal leg has a distal end adjacent the short, distal leg and a free, proximal end and the distancing element is closer to the free proximal end than the distal end with the distancing element maintaining the long, proximal leg at the specific angle.

20. The method of treating a distal radius fracture according to claim 1 wherein the step of fastening the short, distal leg comprises directing a plurality of fasteners through the short, distal leg and into the bone fragment and the step of fastening the long, proximal leg comprises directing a plurality of fasteners through the long, proximal leg and into the radius shaft.
